(19) United States
(12) Patent Application Publication
(10) Pub. No.: US 2026/0327649 A1
(43) Pub. Date: Oct. 22, 2026

(54) COMPOSITION AND METHOD OF UNIVERSAL PSEUDOTYPED RETROVIRUSES

(71) Applicant: The Board of Trustees of the Leland Stanford Junior University, Stanford, CA (US)

(72) Inventors: Lei S. Qi, Stanford, CA (US); Michael Chavez, Stanford, CA (US); Paul B. Finn, Stanford, CA (US)

(73) Assignee: The Board of Trustees of the Leland Stanford Junior University, Stanford, CA (US)

(21) Appl. No.: 19/108,985

(22) PCT Filed: Sep. 12, 2023
(86) PCT No.: PCT/US2023/073983

§ 371 Date: Mar. 5, 2025

Related U.S. Application Data
(60) Provisional application No. 63/405,720, filed on Sep. 12, 2022.

(51) Int. Cl. C12N 15/86; C07K 14/005; C07K 16/28

(52) U.S. Cl. CPC C12N 15/86; C07K 14/005; C07K 16/2803; C07K 2317/569; C07K 2317/622; C12N 2740/15022; C12N 2740/15023; C12N 2740/15043; C12N 2740/15052; C12N 2760/20222

(57) ABSTRACT

Provided herein are retroviral vector systems useful for producing universal pseudotyped retroviruses for cell and gene therapies. The vector systems include an envelope plasmid and a packaging plasmid, where at least one of these plasmids encodes a binding moiety that directly binds a target cell through a cell binding domain, or that indirectly binds a target cell through an antibody binding domain. Also provided are related retrovirus-packaging cells, retroviruses, virus-like particles, and methods for their production and use in preventing or treating a disease.

### CROSS-REFERENCES TO RELATED APPLICATIONS

The present application claims priority to U.S. Provisional Application No. 63/405,720, filed Sep. 12, 2022, the full disclosure of which is incorporated by reference in its entirety for all purposes.

### BACKGROUND

Modern developments in cell and gene therapy are revolutionizing approaches to treating diseases. Gene therapies may be used to correct genetic disorders by editing the genome, e.g., through the use of Clustered Regularly Interspaced Short Palindromic Repeats (CRISPR) gene editing sequences. Gene therapies also may address genetic disorders by introducing new corrective genetic information, e.g. through the use of clinical-grade viruses. Cell therapies may be applied to reprogram cells, directing the human body's natural, complex functionality toward new targets. This opens an enormous space of possible therapeutic modalities that were previously unavailable using old paradigms. Both cell and gene therapies rely on successful delivery of therapeutic genetic payloads to precisely targeted cells or tissues. Many of these therapies also need the delivered genetic information to be directly integrated into the genome, enabling long-term, heritable, and stable expression of the gene.

Retroviruses akin to the human immunodeficiency virus (HIV) have been adapted to accomplish the delivery and integration of genetic payloads associated with cell and gene therapies. Cancer cell lines can be leveraged as hosts to make these retroviruses by transfecting these host cell lines with multiple plasmids or vectors. A transfer vector encodes the genome of the retrovirus to be produced by the host cell. The transfer vector thus includes the genetic payload, e.g., therapeutic genes or gene circuits, that is to be integrated into the genome of a cell targeted by the retrovirus. A packaging plasmid encodes the viral Gag-Pol proteins that enable both production of the retrovirus within the host cell, and integration of the genetic payload into the host cell genome. An envelope plasmid encodes a binding protein that enables the retrovirus to target host cells and begin the viral entry process.

Once cell lines have created these retroviruses, the viruses can be leveraged to introduce new genetic material into cells. The envelope proteins direct viruses toward a surface receptor on the host cell, binding the two together and beginning the viral entry process. Once bound, proteins encoded on the transfer plasmid fuse the cell and virus together, releasing the contents of the virus into the cell's cytoplasm. In a complex set of steps, the viral genome is reverse transcribed from RNA to DNA and is brought into the nucleus, where a viral integrase semi-randomly integrates the viral DNA into the genome. The introduced payload can then divide with the cell and is subject to all other genomic processes, creating a long-term change that enables both cell and gene therapy.

However, major challenges exists with these applications of retroviruses, massively reducing their utility in more advanced cell and gene therapies. These therapies increasingly require selective transduction to only desired cell types, and achieving such selectivity remains extremely difficult for multiple reasons.

First, viral envelope proteins often do not target specific cells of interest. The most ubiquitous envelope protein used with retroviruses is VSV-G, which binds LDL-receptor proteins. This family of surface proteins, though, are not present or adequately expressed on many therapeutically important cell types. For example, Natural Killer (NK) cells are a type of immune cells having extremely exciting potentials as cures for cancers, autoimmune disorders, and diseases of aging. Unfortunately, Natural Killer cells do not express LDL and are therefore difficult to adapt into an engineered cell therapy. Use of other envelope proteins such as RD114 (that binds to RDR surface proteins) or BaEV (that binds to ASCT surface proteins) can at least somewhat overcome this issue, but often at the cost of less efficient viral manufacturing or more limited knowledge of the envelope protein binding partner.

Second, viral envelope proteins lack selectivity. The binding partners of these proteins are not restricted to being expressed by a specific cell type. As a result, retroviruses expressing a particular envelope protein generally cannot target a specific tissue when delivered in situ. Retroviruses also typically cannot target a specific cell type in a mixture when the viruses are administered ex vivo. As an example, currently available technology does not allow the engineering of only the T cells in a peripheral blood mononuclear cell (PBMC) culture by using a retrovirus as an engineering tool.

In view of these observations and results, there is a need in the art for new systems and techniques improving the usefulness of retroviruses, especially in the fields of gene and cell therapy. In particular, new developments are needed to expand the space of cell types that can be engineered by retroviruses, while also making the viruses highly selective to a cell or tissue of interest. The disclosure herein provides a set of solutions to address these challenges, and offers associated and other advantages.

### BRIEF SUMMARY

In general, provided herein are systems, materials, and methods related to retroviruses engineered to be decorated with binding moieties allowing the viruses to bind directly or indirectly to target cells of interest. By leveraging certain types of binding moieties and their binding partners, such as antibodies, antibody fragments, and endogenous or engineered ligands, the provided approaches and compositions enable the design and use of retroviruses with greater specificity and flexibility than was previously possible. The synthetic materials and related methods disclosed herein thus provide useful tools for broad applications including those in the fields of gene therapy and cell engineering.

In one aspect, the disclosure provides a retroviral vector system including an envelope plasmid, a packaging plasmid, and a transfer plasmid. The envelope plasmid encodes a viral membrane fusion protein of a retrovirus. The packaging plasmid encodes Gag-Pol proteins of the retrovirus. The transfer plasmid includes one or more genes of interest for transfer from the retrovirus to a target cell. One or both of the envelope plasmid and the packaging plasmid further encodes a binding moiety. The binding moiety directly or indirectly binds a surface feature of the target cell.

In another aspect, the disclosure provides a retrovirus-packaging cell. The retrovirus-packaging cell includes any of the retroviral packing systems disclosed herein.

In another aspect, the disclosure provides a retrovirus including a viral membrane fusion protein, a viral genome, and a binding moiety. The viral genome includes one or more genes of interest for transfer from the retrovirus to a target cell. The binding moiety binds a ligand that includes an antibody, an antibody mimetic, a single-chain variable fragment (scFv), or a derivative or fragment thereof. The ligand binds a surface feature of the target cell.

In another aspect, the disclosure provides a virus-like particle including a binding moiety. The binding moiety binds a ligand that includes an antibody, an antibody mimetic, a single-chain variable fragment (scFv), or a derivative or fragment thereof. The ligand binds a surface feature of the target cell.

In another aspect, the disclosure provides a method for producing a retrovirus. The method includes transfecting a host cell with any of the retroviral vector systems disclosed herein.

In another aspect, the disclosure provides a method for preventing or treating a disease in a subject. The method includes administering to the subject any of the retroviral vector systems, retrovirus-packaging cells, retroviruses, or virus-like particles disclosed herein.

### BRIEF DESCRIPTION OF THE DRAWINGS

FIG. 1 is an illustration of a retroviral vector system, retrovirus-packaging cells, and retroviruses in accordance with provided embodiments.

FIG. 2 is a schematic illustration of an envelope plasmid encoding a binding moiety in accordance with a provided embodiment.

FIG. 3 presents results from an example demonstrating lymphocyte transduction by a provided retrovirus having a CD7-targeting nanobody as a binding moiety.

FIG. 4 is a graph plotting the transduction of Natural Killer (NK) cells by a provided retrovirus or by a comparative lentivirus.

FIG. 5 is an illustration of a universal pseudotyped retrovirus in accordance with a provided embodiment.

FIG. 6 is a graph plotting the transduction of Jurkat cells at different concentrations of (1) a provided retrovirus having monomeric streptavidin (mSA) as a binding moiety, and (2) a CD71 antibody conjugated to biotin.

FIG. 7 is a graph plotting the transduction of Jurkat cells at different concentrations of (1) a provided retrovirus having an anti-fluorescein isothiocyanate (αFITC) binding moiety, and (2) a CD71 antibody conjugated to FITC.

FIG. 8 is a graph plotting the magnitude of retrovirus-delivered mCherry expression in the Jurkat cells of FIG. 6.

FIG. 9 is a graph plotting the magnitude of retrovirus-delivered mCherry expression in the Jurkat cells of FIG. 7.

FIG. 10 is a graph plotting the transduction of Jurkat cells by a provided retrovirus having a FITC single-chain variable fragment (scFv) binding moiety, where the Jurkat cells have been engineered with antibodies against lymphocyte markers (CD7) or T cell markers (CD3).

FIG. 11 is a graph plotting the transduction of Raji cells by a provided retrovirus having a FITC single-chain variable fragment (scFv) binding moiety, where the Raji cells have been engineered with antibodies against B cell marker CD19 or B cell marker CD20.

FIG. 12 is a graph plotting the transduction of primary T cells by a provided retrovirus having a FITC single-chain variable fragment (scFv) binding moiety, where the T cells have been engineered with antibodies against subtype marker CD4 or subtype marker CD8.

FIG. 13 is a graph plotting the specific transduction of CD4⁺ T cells, and not CD8⁺ T cells, by a provided retrovirus having a FITC binding moiety conjugated to a CD4 antibody.

FIG. 14 is a graph plotting the specific transduction of CD8⁺ T cells, and not CD4⁺ T cells, by a provided retrovirus having a FITC binding moiety conjugated to a CD8 antibody

### DETAILED DESCRIPTION

### I. General

The present disclosure generally provides materials and methods related to retroviruses and virus-like particles engineered to possess modular binding moieties on their surfaces. The particular binding moieties described herein decorate the retroviruses and virus-like particles, and allow them either to bind directly to cells of interest, or to bind an antibody, antibody fragment, or other ligand that binds to the cells. The moieties can advantageously bind to readily available off-the-shelf antibodies, to easily customized antibodies, or to surface features already present on the cells being targeted.

The modular plug-and-play manner in which the binding moieties can be designed or selected offers several significant advantages over existing retrovirus methodologies. For example, the transduction space available to the retroviruses and virus-like particles can be increased to include additional cell types that cannot be transduced by traditional lentivirus approaches. Importantly, these additional cell types include many, such as Natural Killer (NK) cells, that are prime candidates for targeting in developing cellular and genetic therapies. The binding moieties also can enable the universal pseudotyping of a retrovirus or virus-like particle, such that one retrovirus or virus-like particle design can be conjugated with any antibody to target any cell.

While the scope of cells that can be targeted for transduction with the provided tools and techniques is therefore broad, the targeting can also advantageously be more specific than can be achieved with other retrovirus approaches. By tailoring the binding moieties to recognize particular binding partners, off-target transduction can be minimized or substantially eliminated. This reduction of undesirable non-specific targeting can provide important benefits in, for example, therapeutic applications, where non-specific targeting can lead to detrimental side effects. This important cell specificity of the provided retroviruses and virus-like particles can be easily switchable from one target to another due to the modular nature of the binding moieties and their activities.

The binding moieties for the provided retroviruses and virus-like particles are encoded on the packaging and/or envelope plasmids of the viruses or particles. As a result, the genes encoding the binding moieties beneficially do not become integrated into host cells producing the viruses or particles or targeted for transduction. Additionally, the systems for generating the provided retroviruses and virus-like particles can include a minimal number of plasmids, simplifying processes and increasing efficiency. For example, a plasmid system for generating a provided retrovirus can include as few as three plasmids: the packaging and envelope plasmids, at least one of which will encode the binding moiety, and a transfer plasmid that carries the genetic material to be transferred to the target cell.

The production of the provided retroviruses and virus-like particles is also advantageously facile and can easily be incorporated into standard viral production procedures. For example, viral producing cells, such as HEK293T cells, can be transfected with a transfer plasmid encoding a viral genome, an envelope plasmid encoding the viral envelope, and one or more packaging plasmids encoding the viral Gag-Pol proteins. The envelope and/or packaging plasmid in such a system can contain the binding moiety. Viral producing cells containing these plasmids then generate functional viruses that present the binding moieties on their surface. In some instances, antibodies can be incubated with purified retrovirus to conjugate the antibody to the virus. The antibody then can direct the retrovirus to the target cells.

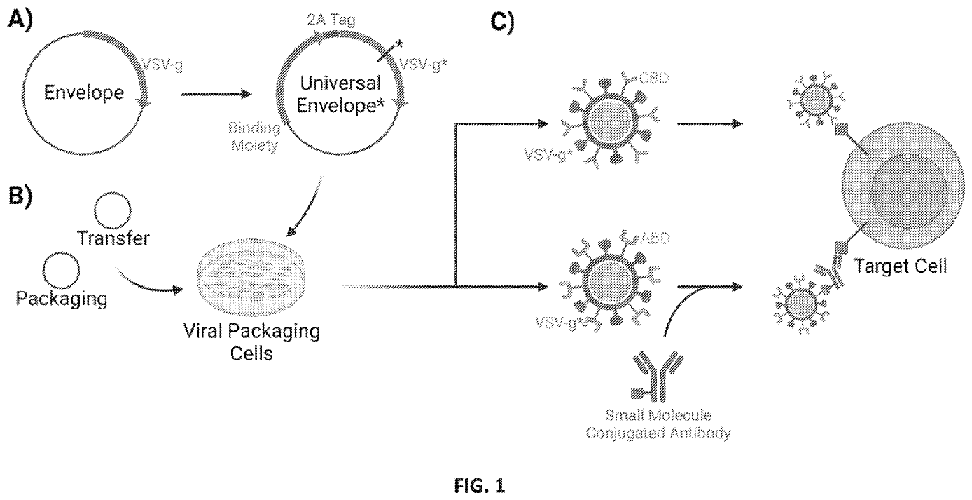

FIG. 1 illustrates several exemplary aspects of the provided retroviral vector systems, retrovirus-packaging cells, and methods for targeting cells. Universal retroviruses can be created by engineering the packaging plasmid or, as shown in panel (A) of the figure, the envelope plasmid, with a binding moiety. The binding moiety can thus include a binding domain directly fused to a transmembrane domain. The binding moiety can be encoded directly upstream or downstream of the envelope or Gag-Pol proteins, and can be separated from the protein post-translationally by a 2A tag. As also shown in the figure, the cognate envelope protein, such as VSV-G, can further be mutated to no longer bind to its cognate ligand.

Panel (B) of FIG. 1 illustrates transfection of viral packaging cells with the engineered envelope plasmid together with transfer and packaging plasmids. These transfected cells then generate the retroviruses decorated with the binding moiety as shown in panel (C). The binding moiety can contain cither a cell binding domain (CBD, top) or an antibody/antibody-fragment/ligand binding domain (ABD, bottom). In the case where the binding moiety contains a CBD, the binding moiety directs the virus toward a specific surface protein of the target cell, enabling the virus to transduce this particular cell type. In the case where the binding moiety contains an ABD, the binding moiety binds a secondary binder such as an antibody, and the combined virus and antibody together transduce specific targeted cell types.

### II. Definitions

Unless defined otherwise, technical and scientific terms used herein have the same meaning as commonly understood by one of ordinary skill in the relevant art.

As used herein, the term “retrovirus” refers to a member of the Retroviridae virus family. A retrovirus has a single-stranded, diploid, positive-sense RNA genome that is reverse-transcribed into a DNA intermediate that can then be incorporated into a host cell genome. Retroviridae-derived viruses are generally enveloped particles with a diameter of 80-120 nm. Retroviral vectors or plasmids can be replication-deficient viral particles derived from the Retroviridae virus family. The plasmids can contain group-specific antigen (Gag) and Pol proteins, a single-stranded RNA genome, and envelope proteins. Retroviral plasmids can also include psi elements and long terminal repeats (LTRs) that can be required for efficient packing and reverse transcription in DNA. Retroviruses include alpharetroviruses, gammaretroviruses, and lentiviruses. Representative species of lentiviruses include the human immunodeficiency virus (HIV). Representative species of gammaretroviruses include murine leukemia virus and the feline leukemia virus.

Integrase-deficient retroviruses and retroviral vectors cannot integrate the retroviral vector genome in the host cell genome. Integrase-deficient retroviral vectors or plasmids can be derived from conventional retroviral vectors, and lack, or contain a mutated form of, the retroviral integrase. Upon entry into the host cell, the retroviral vector genome of an integrase-deficient retrovirus is reverse-transcribed in the cytoplasm, and delivered into the nucleus, but not stably integrated into the host cell genome.

As used herein, the term “transduction” refers to the processes of a virus entering a host cell and delivering an RNA genome, and the expression of a thus-delivered gene of interest by the host cell.

As used herein, the terms “virus-like particle” and “VLP” refer to particles that resemble viruses, but are not infecting or transducing because they contain no viral genetic material encoding the proteins of the virus-like particle. The expression of viral structural proteins, such as envelope or capsid proteins, can result in the assembly of virus-like particles. Virus-like particles can be used to deliver proteins and/or nucleic acids to the cytoplasm of target cells.

As used herein, the term “plasmid” refers to a circular, double-stranded DNA containing one or more sequences of interest, for example, sequences encoding one or more particular proteins. In some embodiments, a plasmid can further include regulatory sequences or other genetic elements that are operatively linked to a sequence encoding a particular protein.

As used herein, the terms “viral membrane fusion protein, “membrane fusion protein,” “fusion protein,” and “fusogen” refer to a polypeptide that causes or enhances fusion of biological membranes, e.g., a viral envelope and a cell wall. A viral membrane fusion protein can be a transmembrane protein or a functional fragment or derivative thereof.

As used herein, the term “cell” generally refers to a biological cell. A cell can be the basic structural, functional and/or biological unit of a living organism. A cell can originate from any organism having one or more cells. Some non-limiting examples include: a prokaryotic cell, eukaryotic cell, a bacterial cell, an archaeal cell, a cell of a single-cell eukaryotic organism, a protozoa cell, a cell from a plant (e.g., cells from plant crops, fruits, vegetables, grains, soy bean, corn, maize, wheat, seeds, tomatoes, rice, cassava, sugarcane, pumpkin, hay, potatoes, cotton, cannabis, tobacco, flowering plants, conifers, gymnosperms, ferns, clubmosses, homworts, liverworts, mosses), an algal cell, (e.g., Botryococcus braunii, Chlamydomonas reinhardtii, Nannochloropsis gaditana, Chlorella pyrenoidosa, Sargassum patens, and the like), seaweeds (e.g., kelp), a fungal cell (e.g., a yeast cell, a cell from a mushroom), a cell from an invertebrate animal (e.g., fruit fly, cnidarian, echinoderm, nematode, mollusk, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), etc. Sometimes a cell does not originate from a natural organism (e.g., a cell can be a synthetically made, sometimes termed an artificial cell).

As used herein, the term “variant,” as used in the context of polypeptides described herein, refers to polypeptides having a high degree of structural similarity to one another, with structural differences resulting from differences in polynucleotides encoding the polypeptide variants. Polypeptide variants may have amino acid sequences that are at least 80% similar to one another (% identity), e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% similar to one another (% identity). Polypeptide variants may have the same biological functions as one another. For example, where polypeptide variants are enzymes, the polypeptide variants may each catalyze the same reaction. Alternatively, a variant of an original polypeptide may be specifically configured or selected to lack the biological activity or function of the original polypeptide.

As used herein, the term “antibody” refers to a polypeptide of the immunoglobulin family or a polypeptide comprising fragments of an immunoglobulin that is capable of noncovalently, reversibly, and in a specific manner binding to an epitope of a corresponding antigen. The term includes, but is not limited to, polyclonal or monoclonal antibodies of the isotype classes IgA, IgD, IgE, IgG, and IgM, derived from human or other mammalian cells, including natural or genetically modified forms such as humanized, human, single-chain, chimeric, synthetic, recombinant, hybrid, mutated, grafted, and in vitro generated antibodies. The term encompasses conjugates, including but not limited to proteins containing an immunoglobulin moiety (e.g., chimeric or bispecific antibodies or single chain Fv's (scFv's)), and fragments, such as Fab, F(ab′)2, Fv, scFv, Fd, dAb and other compositions.

As used herein, the terms “single-chain variable fragment,” “single chain Fv,” and “scFv” refer to an antibody in which the variable domains of the heavy chain and of the light chain of a traditional two chain antibody have been joined to form one chain. Typically, a linker peptide is inserted between the two chains to allow for proper folding and creation of an active binding site.

As used herein, the terms “nanobody” or “single-domain antibody” refer to an antibody fragment comprised of a single monomeric variable antibody domain, having a molecular weight of less than 20 kDa, and able to bind selectively to a specific antigen.

As used herein, the term “epitope” refers to the localized site on the antigen that is recognized and bound by the antibody. Protein epitopes can include a few amino acids or portions of a few amino acids, e.g., 5 or 6 or more, or 20 or more amino acids or portions of those amino acids. Epitopes can also include non-protein components, e.g., nucleic acid (e.g., RNA or DNA), carbohydrate, lipid, or a combination thereof. The epitope can be a three-dimensional moiety. Thus, for example, when the target is a protein target, the epitope can include consecutive amino acids, or amino acids from different parts of the protein that are brought into proximity by protein folding (e.g., a discontinuous epitope). The same is true for other types of target molecules, such as DNA and chromatin, which form three-dimensional structures.

As used herein the terms “specifically (or selectively) binds” or when referring to an antibody interaction, “specifically (or selectively) immunoreactive with,” refers to a binding reaction between two molecules that is at least two times the background and more typically more than 10 to 100 times background molecular associations under physiological conditions. When using one or more detectable binding agents that are proteins, specific binding is determinative of the presence of the protein in a heterogeneous population of proteins and other biologics. Thus, under designated immunoassay conditions, the specified antibodies bind to a particular protein sequence, thereby identifying its presence.

Specific binding to an antibody under such conditions requires an antibody that is selected for its specificity for a particular protein. For example, antibodies raised against a particular protein, polymorphic variants, alleles, orthologs, and conservatively modified variants, or splice variants, or portions thereof, can be selected to obtain only those polyclonal antibodies that are specifically immunoreactive with the protein of interest and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity). Methods for determining whether two molecules specifically interact are disclosed herein, and methods of determining binding affinity and specificity are well known in the art (see, for example, Harlow and Lane, Antibodies: A laboratory manual (Cold Spring Harbor Laboratory Press, 1988); Friefelder, “Physical Biochemistry: Applications to biochemistry and molecular biology” (W. H. Freeman and Co. 1976)).

As used herein, the terms “switch receptor” and “chimeric switch receptor” refer to a molecule designed to switch a negative signal transduction signal into a positive signal. The switch receptor can be a chimeric protein comprising a first protein or fragment thereof associated with a negative signal, and a second protein or fragment thereof associated with a positive signal. Examples of proteins associated with a negative signal include, without limitation, CTLA-4, PD-1, BTLA, TIM-3 and the like. Examples of proteins associated with a positive signal include, without limitation, CD28, ICOS, 4-1BB, TGFβR and the like

As used herein, the term “selectable marker” refers to a gene that encodes a protein that allows the cell expressing the gene to be identified and/or isolated from other cells in a population. Selectable markers include, but are not limited to, genes that encode drug resistance, fluorescence, and essential genes for growth in limiting conditions.

As used herein, the term “subject” refers to a vertebrate, and preferably to a mammal. Mammalian subjects for which the provided composition is suitable include, but are not limited to, mice, rats, simians, humans, farm animals, sport animals, and pets. In some embodiments, the subject is human. In some embodiments, the subject is male. In some embodiments, the subject is female. In some embodiments, the subject is an adult. In some embodiments, the subject is an adolescent. In some embodiments, the subject is a child. In some embodiments, the subject is above 10 years of age, e.g., above 20 years of age, above 30 years of age, above 40 years of age, above 50 years of age, above 60 years of age, above 70 years of age, or above 80 years of age. In some embodiments, the subject is less than 80 years of age, e.g., less than 70 years of age, less than 60 years of age, less than 50 years of age, less than 40 years of age, less than 30 years of age, less than 20 years of age, or less than 10 years of age.

As used herein, the term “administering” refers to oral administration, administration as a suppository, topical contact, parenteral, intravenous, intraperitoneal, intramuscular, intralesional, intranasal or subcutaneous administration, intrathecal administration, or the implantation of a slow-release device e.g., a mini-osmotic pump, to the subject.

As used here, the terms “treat”, “treating” and “treatment” refer to a procedure resulting in any indicia of success in the elimination or amelioration of an injury, pathology, condition, or symptom (e.g., pain), including any objective or subjective parameter such as abatement; remission; diminishing of symptoms or making the symptom, injury, pathology or condition more tolerable to the patient; decreasing the frequency or duration of the symptom or condition; or, in some situations, preventing the onset of the symptom. The treatment or amelioration of symptoms can be based on any objective or subjective parameter; including, e.g., the result of a physical examination.

As used herein, the terms “pharmaceutically acceptable excipient” and “pharmaceutically acceptable carrier” refer to a substance that aids the administration of an active agent to and absorption by a subject and may be included in the compositions of the present disclosure without causing a significant adverse toxicological effect on the subject. Non-limiting examples of pharmaceutically acceptable excipients and carriers include water, NaCl, normal saline solutions, normal sucrose, normal glucose, binders, fillers, disintegrants, lubricants, coatings, and the like. One of skill in the art will recognize that other pharmaceutically acceptable excipients and carriers are useful in the present disclosure.

As used herein, the term “therapeutically effective amount” refers to an amount or dose of a compound, composition, or formulation that produces therapeutic effects for which it is administered. The exact amount or dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

As used herein, the term “vaccine” refers to a composition comprising at least one antigen or immunogen, or comprising a nucleic acid molecule encoding at least one antigen or immunogen, in a pharmaceutically acceptable carrier, that is useful for inducing an immune response against the antigen or immunogen in a subject, for the purpose of improving immunity against a disease and/or infection in the subject.

As used herein, the singular forms “a,” “an,” and “the” include plural referents unless the content clearly dictates otherwise. Thus, for example, reference to “a polymer” optionally includes a combination of two or more polymers, and the like.

As used herein, the term “and/or” refers to and encompasses any and all possible combinations of one or more of the associated listed items, as well as the lack of combinations when interpreted in the alternative (“or”).

As used herein, the terms “including,” “comprising,” “having,” “containing,” and variations thereof, are inclusive and open-ended and do not exclude additional, unrecited elements or method steps beyond those explicitly recited. As used herein, the phrase “consisting of” is closed and excludes any element, step, or ingredient not explicitly specified. As used herein, the phrase “consisting essentially of” limits the scope of the described feature to the specified materials or steps and those that do not materially affect the basic and novel characteristics of the disclosed feature.

### III. Retroviral Vector Systems

In one aspect of the disclosure, a retroviral vector system is provided. The vector system includes plasmids that can be used to transform a retrovirus-packaging cell, enabling the cell to produce retroviruses according to the contents of the vector system. The disclosed retroviral vector system can therefore provide many of the surprising advantages discussed herein, particularly when used in therapeutic applications such as gene or cell therapies. For example, the retroviral vector system can be used as a tool for producing retroviruses engineered to specifically and/or selectively transduce one or more particular cell types or populations. In certain aspects, the engineered retroviruses produced using the retroviral vector system can also advantageously have a cell specificity that is easily switchable. This switchable specificity can be realized by, for example, using facile procedures to alter the binding moieties of the retroviruses or to alter a secondary binder, e.g., an antibody, to which the retrovirus binding moieties conjugate.

The provided retroviral vector systems include one or more envelope plasmids, one or more packaging plasmids, and one or more transfer plasmids. An advantage of the provided vector systems is that a small number of plasmids can be effective in transfecting a virus-packing cell to enable the cell to produce a functional retrovirus having the binding moiety disclosed herein. In some embodiments, the retroviral vector system includes only one envelope plasmid. In some embodiments, the retroviral vector system includes only one packaging plasmid. In some embodiments, the retroviral vector system includes only one transfer plasmid. In some embodiments, the retroviral vector system includes only one envelope plasmid and only one packaging plasmid. In some embodiments, the retroviral packaging system includes only one envelope plasmid and only one transfer plasmid. In some embodiments, the retroviral vector system includes only one packaging plasmid and only one transfer plasmid. In some embodiments, the retroviral vector system includes only one envelope plasmid, only one packaging plasmid, and only one transfer plasmid.

The envelope plasmid of the provided retroviral vector system generally encodes a viral membrane fusion protein of the retrovirus to be produced by a virus-packaging cell transfected with the vector system. The packaging plasmid of the retroviral vector system generally encodes Gag-Pol proteins of the retrovirus. The transfer plasmid of the retroviral vector system generally encodes one or more genes of interest for transfer from the retrovirus to a target cell.

At least one plasmid of the provided retroviral vector system encodes a binding moiety that directly or indirectly binds a surface feature of a target cell. Encoding the binding moiety with one of the plasmids of the vector system advantageously ensures that all retroviruses produced using the system will include the binding moiety. In some embodiments, only one plasmid of the retroviral vector system encodes the binding moiety. In some embodiments, two or more different plasmids of the retroviral vector system encode the binding moiety. In some embodiments, each different plasmid of the retroviral vector system encodes the binding moiety.

For certain applications, it can be desirable to only encode the binding moiety with one or both of the envelope plasmid and the packaging plasmid, and not with the transfer plasmid. In this way, the binding moiety does not become integrated into the genome of host cells targeted by retroviruses produced using the vector system. In some embodiments, only the one or more envelope plasmids encode the binding moiety. In some embodiments, only the one or more packaging plasmids encode the binding moiety. In some embodiments, the envelope plasmids and packaging plasmids each encode the binding moiety, while the transfer plasmid does not.

The binding moiety of the provided retroviral vector system generally includes an extramembrane domain, where the extramembrane domain is a binding domain that recognizes a surface feature of a target cell, or that recognizes a secondary binding partner that recognizes a surface feature of the target cell. In some embodiments, the binding moiety includes a transmembrane domain that positions the binding moiety in the envelope of the retrovirus. In some embodiments, the extramembrane domain of the binding moiety is fused to the transmembrane domain of another viral envelope protein.

The binding moiety can be encoded directly upstream of an envelope protein on the envelope plasmid of the provided retroviral vector system. The binding moiety can be encoded directly downstream of an envelope protein on the envelope plasmid (FIG. 2). The binding moiety can be encoded directly upstream of a Gag-Pol protein on the packaging plasmid of the retroviral vector system. The binding moiety can be encoded directly downstream of a Gag-Pol protein on the packaging plasmid. In some embodiments, the binding moiety is post-translationally separated from the adjacent protein of the plasmid, e.g., with a 2A tag.

In some embodiments, the binding moiety of the provided retroviral vector system is designed or selected to bind with a ligand, where the ligand is designed or selected to bind with a target cell, e.g., to a surface feature of the target cell (FIG. 1). In some embodiments, the ligand bound by the binding moiety includes an antibody, an antibody mimetic, a single-chain variable fragment (scFv), or a derivate or fragment thereof. In such embodiments, the binding moiety can be described as including an antibody binding domain (ABD).

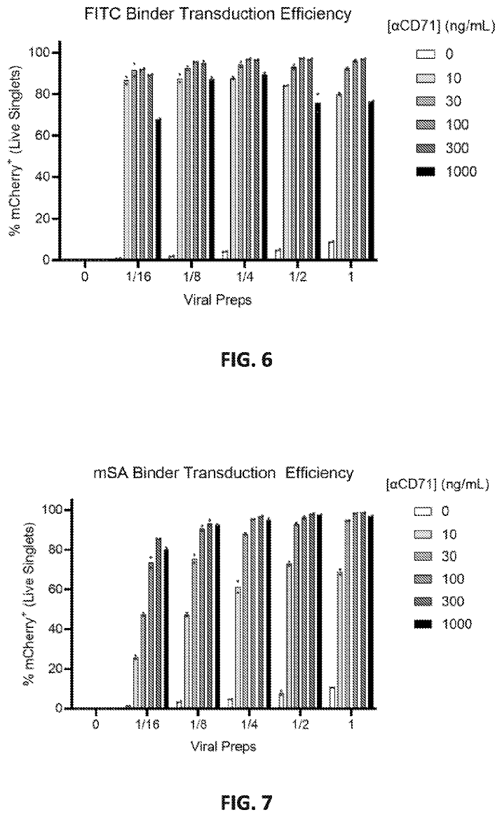

In some embodiments, the ligand is a conjugated derivative of an antibody, an antibody mimetic, or an scFv, where the ligand is conjugated to a small molecule recognized by the binding moiety (FIG. 5). Using this approach, a retroviral vector system can be engineered such that it can be used to produce universal pseudotyped retroviruses. These universal pseudotyped retroviruses each include a binding moiety that binds to a particular small molecule, and any conjugate between a ligand and the small molecule can easily be created so that the ligand is compatible with the binding moiety having an affinity for the particular small molecule of the conjugate. In this way, a single universal design of a provided retrovirus, e.g. a retrovirus produced using the provided retroviral vector system, can specifically bind to a particular cell type if a ligand-small molecule conjugate specific for that cell type is applied.

In some embodiments, the binding moiety includes a fluorescein isothiocyanate (FITC)-binding domain, and the ligand is FITC-conjugated. For example, the binding moiety can include an anti-fluorescein scFv or fluorescein-binding anticalin. The ligand recognized by this binding moiety can be, for example, a FITC-conjugated antibody. Because of the availability of FITC-conjugated antibodies specific for a variety of different cell-specific antigens, a single design of a universal pseudotyped retrovirus with a FITC-binding moiety can thus specifically transduce a variety of cell types.

In some embodiments, the binding moiety includes a biotin-binding domain, and the ligand is biotin-conjugated. For example, the binding moiety can include an anti-biotin scFv, biotin-binding anticalin, or an avidin-family protein, e.g., avidin or streptavidin. The ligand recognized by this binding moiety can be, for example, a biotin-conjugated antibody. Because of the availability of biotin-conjugated antibodies specific for a variety of different cell-specific antigens, a single design of a universal pseudotyped retrovirus with a biotin-binding moiety can thus specifically transduce a variety of cell types.

The ligands recognized by an antibody binding domain of a provided binding moiety can themselves bind to a wide variety of surface features of target cells. Many off-the-shelf antibodies suitable for use with the provided materials and methods are available, where the available antibodies have different specificity and selectivity for various antigens and epitopes. Still other antibodies and antibody conjugates can be developed for binding to further target cell surface features. For example, and without limitation, the ligands can be used to indirectly bind the provided binding moiety to one or more of CD3, CD4, CD7, CD8, CD19, CD20, CD56, CD71, or CTLA4.

In some embodiments, the binding moiety of the provided retroviral vector system is designed or selected to bind directly with a target cell, e.g., to a surface feature of the target cell (FIG. 1). In such embodiments, the binding moiety can be described as a including a cell binding domain (CBD). In some embodiments, the cell binding domain includes an antibody, an antibody mimetic, an scFv, a nanobody, another ligand, or a derivative or fragment thereof. As with binding moieties including antibody binding domains, binding moieties including cell binding domains can also bind to a wide variety of surface features of target cells. For example, and without limitation, the cell binding domain can be designed or selected to bind the provided binding moiety to one or more of CD3, CD4, CD7, CD8, CD19, CD20, CD56, CD71, or CTLA4.

A wide variety of cell types can be targeted by the provided retroviral vector systems. In some embodiments, the target cell is an immune cell, including any cell that is involved in an immune response. For example, the targeting of immune cells such as optionally allogenic natural NK cells, iPSC-derived NK cells, and/or macrophage cells can greatly facilitate treatment of solid tumor while avoiding side effects. In this way the provided retroviral vector systems can mediate precise activation of immunity at a precise location (e.g., presence of a local tumor signal) and at a specific time point.

In some embodiments, the target cell includes granulocytes such as basophils, cosinophils, and neutrophils; mast cells; monocytes which can develop into macrophages; antigen-presenting cells such as dendritic cells; and lymphocytes such as natural killer cells (NK cells), B cells, and T cells. In some embodiments, the target cell is an immune effector cell. An immune effector cell is an immune cell that can perform a specific function in response to a stimulus. In some embodiments, the target cell is an immune effector cell which can induce cell death. In some embodiments, the target cell is a lymphocyte. In some embodiments, the lymphocyte is an NK cell. In some embodiments the lymphocyte is a T cell. In some embodiments, the T cell is an activated T cell. T cells include both naive and memory cells (e.g., central memory or T_{CM}, effector memory or T_{EM} and effector memory RA or T_{EMRA}), effector cells (e.g., cytotoxic T cells or CTLs or Tc cells), helper cells (e.g., Th1, Th2, Th3, Th9, Th7, TFH), regulatory cells (e.g., Treg, and Trl cells), natural killer T cells (NKT cells), tumor infiltrating lymphocytes (TILs), lymphocyte-activated killer cells (LAKs), αβ T cells, γδ T cells, and similar unique classes of the T cell lineage.

T cells can be divided into two broad categories: CD8+ T cells and CD4+ T cells, based on which protein is present on the cell's surface. T cells can carry out multiple functions, including killing infected cells and activating or recruiting other immune cells. CD8+ T cells are referred to as cytotoxic T cells or cytotoxic T lymphocytes (CTLs). CD4+ T cells can be subdivided into four sub-sets-Th1, Th2, Th17, and Treg, with “Th” referring to “T helper cell,” although additional sub-sets may exist. Th1 cells can coordinate immune responses against intracellular microbes, especially bacteria. They can produce and secrete molecules that alert and activate other immune cells, like bacteria-ingesting macrophages. Th2 cells are involved in coordinating immune responses against extracellular pathogens, like helminths (parasitic worms), by alerting B cells, granulocytes, and mast cells. Th17 cells can produce interleukin 17 (IL-17), a signaling molecule that activates immune and non-immune cells. Th17 cells are important for recruiting neutrophils.

The retroviral vector system can be engineered to target cell types that mediate cell differentiation and programming. This can allow site-specific differentiation of cells and tissues to repair or regenerate a damaged or aged body. In some embodiments, the target cell is a stem cell. The target cell can be, for example, an induced pluripotent stem cell (iPSC), an embryonic stem cell (ESC), an adult stem cell, or a mesenchymal stem cell (MSC). In some embodiments, the target cell is a progenitor cell. The target cell can be, for example, a neural progenitor cell, a skeletal progenitor cell, a muscle progenitor cell, a fat progenitor cell, a heart progenitor cell, a chondrocyte, or a pancreatic progenitor cell.

In some embodiments, the provided retroviral vector system is designed to produce retroviruses that target cells of one or more specific tissues. These embodiments can be particularly useful when the retroviral vector system is used to produce retroviruses for in situ engineering or therapy of those specific tissues. In some embodiments, the target cells include muscle cells. In some embodiments, the target cells include neural cells. In some embodiments, the target cells include pancreatic cells, e.g., islets.

In some embodiments, the provided retroviral vector system is designed to produce retroviruses that target two or more different cell types with a different payload delivered to each of the different cell types. For example, a retroviral vector system can include a FITC-binding domain recognizing a FITC-conjugated antibody, where the FITC-conjugated antibody targets a first antigen for delivery of a first payload. The same retroviral vector system can further include a biotin-binding domain recognizing a biotin-conjugated antibody, where the biotin-conjugated antibody targets a second antigen for delivery of a second payload.

The provided retroviral vector systems can be used to deliver any genetically encoded material. The systems, and the retroviruses they create, can be used to deliver, for example, human genes, chimeric and engineered genes, gene editors, and genes from other species. In some embodiments, vector system transfer plasmid genes encode receptors including but not limited to chimeric antigen receptors (CARs) (including first, second, third, or fourth generation receptors), switch receptors, MIMIC receptors, or modified versions thereof. In some embodiments, the transfer vector genes include one or more nucleases such as CRISPR-associated (Cas) proteins or Cas nucleases including type I CRISPR-associated (Cas) polypeptides, type II CRISPR-associated (Cas) polypeptides, type III CRISPR-associated (Cas) polypeptides, type IV CRISPR-associated (Cas) polypeptides, type V CRISPR-associated (Cas) polypeptides, and type VI CRISPR-associated (Cas) polypeptides; zinc finger nucleases (Z. FN); transcription activator-like effector nucleases (TALEN); meganucleases; RNA-binding proteins (RBP); CRISPR-associated RNA binding proteins; recombinases; flippases; transposases; Argonaute (Ago) proteins (e.g., prokaryotic Argonaute (pAgo), archaeal Argonaute (aAgo), eukaryotic Argonaute (eAgo), and Natronobacterium gregoryi Argonaute (NgAgo)); Adenosine deaminases acting on RNA (ADAR); CIRT, PUF, homing endonuclease, or any functional fragment, derivative, or variant thereof. In some embodiments, the transfer vector genes include one or more reporter genes or selectable markers such as a fluorescent protein, an antibiotic resistance gene, or a derivative or fragment thereof.

In some embodiments, the provided transfer plasmid includes more than one gene in one or more polycistronic element. In some embodiments, the retroviral vector system can be used to produce retroviruses that transduce host cells to express more than one gene of a polycistronic element using, for example, IRES or 2A tags. In some embodiments, the transfer plasmid includes one or more regulatory elements such as promoters, introns, enhancers, post-transcriptional elements, or post-translational elements, that can regulate transduced gene expression.

In some embodiments, the viral membrane fusion protein of the provided retroviral vector system envelope plasmid is the vesicular stomatitis virus G (VSV-G) protein, or is an engineered variant thereof. In some embodiments, the viral membrane fusion protein is a membrane fusion protein from feline endogenous virus RD114, or is an engineered variant thereof. In some embodiments, the viral membrane fusion protein is a membrane fusion protein from baboon endogenous virus BaEV, or is an engineered variant thereof.

In some embodiments, the viral membrane fusion protein is an engineered variant that does not bind to a cognate binding partner of the corresponding wild-type viral membrane fusion protein. This can be particularly advantageous for applications where it is important to ensure that a retrovirus produced using the retroviral vector system is reliant on the binding moiety of the vector system for host cell targeting and transduction. In some embodiments, the engineered variant includes one or more, e.g., two or more, point mutations to the sequence of the wild-type viral membrane fusion protein, such that the protein loses its cognate binding ability. In some embodiments, the engineered variant is a truncated mutant of the wild-type viral membrane fusion protein, such that the protein loses its cognate binding ability.

In some embodiments, an integrase enzyme of the provided retroviral vector system is mutated, such that the vector system can be used to produce integrase deficient retroviruses. This can be particularly advantageous for applications aiming to deliver genetic information to a target cell without causing stable integration of that information into the target cell genome.

### IV. Retrovirus-Packaging Cells

In another aspect of the disclosure, a retrovirus-packaging cell is provided. The retrovirus-packaging cell is a cell that has been transformed with any of the provided retroviral vector systems, e.g., any of those described in Section III. The retrovirus-packaging cell can therefore provide many of the surprising advantages discussed herein, particularly when used in therapeutic applications such as gene or cell therapies. For example, the retrovirus-packaging cell can be used as a tool for producing retroviruses engineered to specifically and/or selectively transduce one or more particular cell types or populations. In certain aspects, the engineered retroviruses produced using the retrovirus-packaging cell can also advantageously have a target cell specificity that is easily switchable. This switchable specificity can be realized by, for example, using facile procedures to alter the binding moieties of the retroviruses or to alter a secondary binder, e.g., an antibody, to which the retrovirus binding moieties conjugate.

In another aspect, a population of retrovirus-packaging cells is provided. In some embodiments, each host cell of the population independently includes a retroviral vector system as disclosed herein. A cell culture comprising a population of cells as described herein is also provided. Methods for the culture and production of many cells, including cells of bacterial (for example E. coli and other bacterial strains), animal (especially mammalian), and archacbacterial origin are available in the art. See e.g., Sambrook, supra; Ausubel, ed. (1995) Current Protocols in Molecular Biology, John Wiley & Sons, as well as Freshney (1994) Culture of Animal Cells, a Manual of Basic Technique, 3^{rd} Ed., Wiley-Liss, New York and the references cited therein; Doyle and Griffiths (1997) Mammalian Cell Culture: Essential Techniques John Wiley and Sons, NY; Humason (1979) Animal Tissue Techniques, 4^{th} Ed. W. H. Freeman and Company; and Ricciardelli, et al., (1989) In vitro Cell Dev. Biol. 25:1016-1024.

### V. Retroviruses

In another aspect of the disclosure, a retrovirus is provided. In some embodiments, the retrovirus can be produced using any of the provided retroviral vector systems and/or retrovirus-packaging systems. Accordingly, the retroviruses can provide the surprising advantages discussed herein, including broad applicability, high specificity and selectivity, switchable targeting, and universal pseudotyping.

The provided retrovirus includes a viral membrane fusion protein. The viral membrane fusion protein can be any of those disclosed herein regarding the retroviral vector system. The provided retrovirus further includes a viral genome including one or more genes of interest for transfer from the retrovirus to a target cell. The genes of interest and the target cell can by any of those disclosed herein regarding the retroviral vector system. The provided retrovirus further includes a binding moiety that can be any of those disclosed herein regarding the retroviral vector system. In some embodiments, the binding moiety includes an antibody binding domain (ABD) of any of the types disclosed herein. For example, the binding moiety of the provided retrovirus can include a ligand that is an antibody, an antibody mimetic, a single-chain variable fragment (scFv), or a derivate or fragment thereof, where the ligand binds a surface feature of the target cell.

### VI. Virus-Like Particles

In another aspect of the disclosure, a virus-like particle is provided. The virus-like particle includes many of the same advantages of the provided retroviruses, including broad applicability, high specificity and selectivity, switchable targeting, and universal pseudotyping. Unlike the provided retroviruses, the virus-like particle does not include a viral genome, and therefore cannot be used to integrate delivered genetic material into the genome of a target cell. The virus-like particles therefore offer an attractive alternative to retroviruses where such integration is not desired.

The provided virus-like particles include a binding moiety that can be any of those disclosed herein regarding the retroviral vector system. In some embodiments, the binding moiety includes an antibody binding domain (ABD) of any of the types disclosed herein. For example, the binding moiety of the provided retrovirus can include a ligand that is an antibody, an antibody mimetic, a single-chain variable fragment (scFv), or a derivate or fragment thereof, where the ligand binds a surface feature of the target cell.

### VII. Methods for Preventing or Treating Diseases

In another aspect of the disclosure, a method for preventing or treating a disease is provided. The method includes administering to a subject any of the retroviral vector systems, retrovirus-packaging cells, retroviruses, or virus-like particles disclosed herein, e.g., in Sections III, IV, V, and VI. Pharmaceutical compositions containing retroviral vector systems, retrovirus-packaging cells, retroviruses, or virus-like particles described herein, e.g., in Section IX, can be administered for prophylactic and/or therapeutic treatments. In therapeutic applications, these compositions can be administered to a subject already suffering from a disease or condition, in an amount sufficient to cure or at least partially arrest the symptoms of the disease or condition, or to cure, heal, improve, or ameliorate the condition. Amounts effective for this use can vary based on the severity and course of the disease or condition, previous therapy, the subject's health status, weight, and response to the drugs, and the judgment of the treating physician.

Retroviral vector systems, retrovirus-packaging cells, retroviruses, or virus-like particles described herein can be administered before, during, or after the occurrence of a disease or condition, and the timing of administering the composition can vary. For example, a pharmaceutical compositions can be used as a prophylactic and can be administered continuously to subjects with a propensity to conditions or diseases in order to prevent the occurrence of the disease or condition. The pharmaceutical compositions can be administered to a subject during or as soon as possible after the onset of the symptoms. The administration can be initiated within the first 48 hours of the onset of the symptoms, within the first 24 hours of the onset of the symptoms, within the first 6 hours of the onset of the symptoms, or within 3 hours of the onset of the symptoms. The initial administration can be via any route practical, such as by any route described herein using any formulation described herein. A composition can be administered as soon as is practicable after the onset of a disease or condition is detected or suspected, and for a length of time necessary for the treatment of the disease, such as, for example, from about 1 month to about 3 months. The length of treatment can vary for each subject.

A wide variety of diseases can be prevented or treated using the provided methods. The methods are suitable for broad cell or gene therapy applications to treat, for example, blood or solid cancer, viral infections, bacterial infections, genetic diseases, wound healing, autoimmunity, regenerative medicine, CNS diseases, and anti-aging.

In some embodiments, the prevented of treated disease is a genetic disorder. Disorders suitable for treating with the provided method include, but are not limited to, X-linked severe combined immune deficiency, sickle cell anemia, thalassemia, hemophilia, neoplasia, cancer, age-related macular degeneration, schizophrenia, trinucleotide repeat disorders, fragile X syndrome, prion-related disorders, amyotrophic lateral sclerosis, drug addiction, autism, Alzheimer's disease, Parkinson's disease, cystic fibrosis, blood and coagulation disease or disorders, inflammation, facioscapulohumeral muscular dystrophy, retinitis pigmentosa, Leber congenital amaurosis, glaucoma, immune-related diseases or disorders, metabolic diseases and disorders, liver diseases and disorders, kidney diseases and disorders, muscular/skeletal diseases and disorders, neurological and neuronal diseases and disorders, cardiovascular diseases and disorders, pulmonary diseases and disorders, and ocular diseases and disorders.

In some embodiments, the prevented or treated disease is a cancer. Non-limiting examples of cancers that can be treated with the provided retroviral vector systems, retrovirus-packaging cells, retroviruses, or virus-like particles include Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia. AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloblastoma, Medullocpithelioma, Melanoma, Melanoma, Meningioma, Merkel Cell Carcinoma, Mesothelioma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple Myeloma, Multiple myeloma, Mycosis Fungoides, Mycosis fungoides, Myelodysplastic Disease, Myelodysplastic Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neuroblastoma, Neurofibroma, Neuroma, Nodular melanoma, Non-Hodgkin Lymphoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral Cancer, Oral cancer, Oropharyngeal Cancer, Osteosarcoma, Osteosarcoma, Ovarian Cancer, Ovarian cancer, Ovarian Epithelial Cancer, Ovarian Germ Cell Tumor, Ovarian Low Malignant Potential Tumor, Paget's disease of the breast, Pancoast tumor, Pancreatic Cancer, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, Wilms' tumor, and combinations thereof.

In some embodiments, the prevented or treated disease is a cancerous tumor. The cancerous tumor can be a solid cancerous tumor or a liquid cancerous tumor. The liquid cancerous tumor can be, for example, a lymphoma or a leukemia. A tumor treated with the methods disclosed herein can result in stabilized tumor growth (e.g., one or more tumors do not increase more than 1%, 5%, 10%, 15%, or 20% in size, and/or do not metastasize). In some embodiments, a tumor is stabilized for at least about 1, 2, 3, 4, 5, 6, 7, 8, 9, 10, 11, 12, or more weeks. In some embodiments, a tumor is stabilized for at least about 1, 2, 3, 4, 5, 6, 7, 8, 9, 10, 11, 12, or more months. In some embodiments, a tumor is stabilized for at least about 1, 2, 3, 4, 5, 6, 7, 8, 9, 10, or more years. In some embodiments, the size of a tumor or the number of tumor cells is reduced by at least about 5%, 10%, 15%, 20%, 25%, 30%, 35%, 40%, 45%, 50%, 55%, 60%, 65%, 70%, 75%, 80%, 85%, 90%, 95% or more. In some embodiments, the tumor is completely eliminated, or reduced below a level of detection. In some embodiments, a subject remains tumor free (e.g. in remission) for at least about 1, 2, 3, 4, 5, 6, 7, 8, 9, 10, 11, 12, or more weeks following treatment. In some embodiments, a subject remains tumor free for at least about 1, 2, 3, 4, 5, 6, 7, 8, 9, 10, 11, 12, or more months following treatment. In some embodiments, a subject remains tumor free for at least about 1, 2, 3, 4, 5, 6, 7, 8, 9, 10, or more years after treatment.

One skilled in the art will also appreciate that the provided systems and compositions can be co-administered with other therapeutic agents for the treatment of cancer. Suitable anti-cancer agents for combination therapy include, without limitation, cytotoxins and agents such as antimetabolites, alkylating agents, anthracyclines, antibiotics, antimitotic agents, procarbazine, hydroxyurea, asparaginase, corticosteroids, interferons, radiopharmaceuticals, peptides with anti-tumor activity such as TNF-α, pharmaceutically acceptable salts thereof; derivatives thereof, prodrugs thereof, and combinations thereof. For example, a pharmaceutical composition comprising the provided retroviral vector systems, retrovirus-packaging cells, retroviruses, and/or virus-like particles can be administered to a patient before, during, or after administration of an anti-cancer agent or combination of anti-cancer agents either before, during, or after chemotherapy.

In some embodiments, treatment with the provided retroviral vector systems, retrovirus-packaging cells, retroviruses, and/or virus-like particles results in stable disease, partial remission or complete remission in the subject (e.g., the methods described herein comprise administering to the subject a dose of the provided retroviral vector systems, retrovirus-packaging cells, retroviruses, and/or virus-like particles that kills or otherwise slows the growth or progression of cancer cells and leads to stable disease or to partial or complete remission of the cancer in the subject). In some embodiments, treatment with the provided retroviral vector systems, retrovirus-packaging cells, retroviruses, and/or virus-like particles results in a reduction in metastases of the cancer in the subject (e.g., the methods described herein comprise administering to the subject a dose of the provided retroviral vector systems, retrovirus-packaging cells, retroviruses, and/or virus-like particles that reduces metastases of the cancer in the subject). In some embodiments, treatment with the provided retroviral vector systems, retrovirus-packaging cells, retroviruses, and/or virus-like particles results in a reduction in volume, size, or growth of a tumor in the subject (e.g., the methods described herein comprise administering to the subject a dose of the provided retroviral vector systems, retrovirus-packaging cells, retroviruses, and/or virus-like particles that reduces the volume, size, or growth of a tumor in the subject). In some embodiments, treatment with the provided retroviral vector systems, retrovirus-packaging cells, retroviruses, and/or virus-like particles results in an increased responsiveness of the cancer to a subsequently administered anti-cancer agent (e.g., the methods described herein comprise administering to the subject a dose of the provided retroviral vector systems, retrovirus-packaging cells, retroviruses, and/or virus-like particles that increases responsiveness of the cancer to a subsequently administered anti-cancer agent).

In some embodiments, the prevented or treated disease is an infectious disease. The infectious disease can be, for example, a viral infectious disease. The infectious disease can be, for example, a bacterial infectious disease. In the case of bacterial infections, the innate immune system must recognize specific markers of the microbes in order to clear the pathogen. These pathogen associated molecular patterns are recognized by various receptors, most notably the TLRs that are common to all immune cells and work to activate immune pathways that turn on their bactericidal capacities. In the case of macrophages, recognition of microbial pathogens through a TLR activates their unique ability to engulf the bacteria within themselves and destroy the pathogen by acidification. However, bacteria have mechanisms to evade macrophages by hiding the molecules that cause this activation. For example, during biomedical device implantations such as catheters and pacemakers, bacteria form dense biofilms around themselves to avoid recognition, causing massive infection issues which have inhibited lifesaving technology from moving to the clinic. The provided retroviral vector systems, retrovirus-packaging cells, retroviruses, or virus-like particles can “rewire” these TLRs to recognize the constituents of the biofilm itself as opposed to the bacteria, such that macrophages are activated by the evasion mechanisms, destroying the infection and allowing these devices to be more safely implanted.

In some embodiments, the provided retroviral vector systems, retrovirus-packaging cells, retroviruses, and/or virus-like particles are administered to a subject once, twice, three times, four times, or five times over a course of treatment. Subsequent administration of the provided retroviral vector systems, retrovirus-packaging cells, retroviruses, and/or virus-like particles can occur at defined intervals of time, separated by days, weeks, or months. In some cases, the provided retroviral vector systems, retrovirus-packaging cells, retroviruses, and/or virus-like particles are administered at a subsequent time if a tumor or cancerous cells reappear, continue to grow, or otherwise are not fully treated after the first administration of the provided retroviral vector systems, retrovirus-packaging cells, retroviruses, and/or virus-like particles. In some cases, the provided retroviral vector systems, retrovirus-packaging cells, retroviruses, and/or virus-like particles are administered again at a subsequent time if the subject does not have a complete response to the first treatment, experiences a partial response, a stable response or progressive discase.

In some embodiments, the provided method further includes obtaining a test sample from the subject. The test sample can include, for example, a blood sample, a tissue sample, a urine sample, a saliva sample, a cerebrospinal fluid sample, or a combination thereof. In some embodiments, the provided method further includes determining the level of one or more biomarkers in the obtained test sample. Determining the presence or level of biomarkers(s) can be used to, as non-limiting examples, determine response to treatment or to select an appropriate composition for the prevention or treatment of the disease.

In some embodiments, the provided method further includes comparing the determined level of the one of more biomarkers in the obtained test sample to the level of the one or more biomarkers in a reference sample. The reference sample can be obtained, for example, from the subject, with the reference sample being obtained prior to the obtaining of the test sample, e.g., prior to the administering to the subject of the therapeutically effective amount of the provided materials. In this way, the reference sample can provide information about baseline levels of the biomarkers in the sample before the treatment, and the test sample can provide information about levels of the biomarkers after the treatment.

Alternatively, the reference sample can be obtained, for example, from a different subject, e.g., a subject in which the treatment is not provided according to the provided methods. In this way, the reference sample can provide information about baseline levels of the biomarkers without treatment, and the test sample can provide information about levels of the biomarkers with treatment. The reference sample can also be obtained, for example, from a population of subjects, e.g., subjects in which the treatment is not provided according to the provided method. In this way, the reference sample can provide population-averaged information about baseline levels of the biomarkers without treatment, and the test sample can provide information about levels of the biomarkers with treatment.

The reference sample can also be obtained from an individual or a population of individuals after treatment is provided according to the provided methods, and can serve as, for example, a positive control sample. In some embodiments, the reference sample is obtained from normal tissue. In some embodiments, the reference sample is obtained from abnormal tissue.

Depending on the biomarker, an increase or a decrease relative to a normal control or reference sample can be indicative of the presence of a disease, or response to treatment for a disease. In some embodiments, an increased level of a biomarker in a test sample, and bence the presence of a disease, e.g., an infectious disease or cancer, increased risk of the disease, or response to treatment is determined when the biomarker levels are at least, 1.1-fold, e.g., at least 1.2-fold, at least 1.3-fold, at least 1.4-fold, at least 1.5-fold, at least 1.6-fold, at least 1.7-fold, at least 1.8-fold, at least 1.9-fold, at least 2-fold, at least 3-fold, at least 4-fold, at least 5-fold, at least 6-fold, at least 7-fold, at least 8-fold, at least 9-fold, at least 10-fold, at least 11-fold, at least 12-fold, at least 13-fold, at least 14-fold, at least 15-fold, at least 16-fold, at least 17-fold, at least 18-fold, at least 19-fold, or at least 20-fold higher in comparison to a negative control. In other embodiments, a decreased level of a biomarker in the test sample, and hence the presence of the disease, increased risk of the disease, or response to treatment is determined when the biomarker levels are at least 1.1-fold, e.g., at least 1.2-fold, at least 1.3-fold, at least 1.4-fold, at least 1.5-fold, at least 1.6-fold, at least 1.7-fold, at least 1.8-fold, at least 1.9-fold, at least 2-fold, at least 3-fold, at least 4-fold, at least 5-fold, at least 6-fold, at least 7-fold, at least 8-fold, at least 9-fold, at least 10-fold, at least 11-fold, at least 12-fold, at least 13-fold, at least 14-fold, at least 15-fold, at least 16-fold, at least 17-fold, at least 18-fold, at least 19-fold, or at least 20-fold lower in comparison to a negative control.

The biomarker levels can be detected using any method known in the art, including the use of antibodies specific for the biomarkers. Exemplary methods include, without limitation, polymerase chain reaction (PCR), Western Blot, dot blot, ELISA, radioimmunoassay (RIA), immunoprecipitation, immunofluorescence, FACS analysis, electrochemiluminescence, and multiplex bead assays, e.g., using Luminex or fluorescent microbeads. In some instances, nucleic acid sequencing is employed.

In certain embodiments, the presence of decreased or increased levels of one or more biomarkers is indicated by a detectable signal, e.g., a blot, fluorescence, chemiluminescence, color, or radioactivity, in an immunoassay or PCR reaction, e.g., quantitative PCR. This detectable signal can be compared to the signal from a reference sample or to a threshold value.

In some embodiments, the results of the biomarker level determinations are recorded in a tangible medium. For example, the results of diagnostic assays, e.g., the observation of the presence or decreased or increased presence of one or more biomarkers, and the diagnosis of whether or not there is an increased risk or the presence of a disease, e.g., an infectious disease or cancer, or whether or not a subject is responding to treatment can be recorded, for example, on paper or on electronic media, e.g., audio tape, a computer disk, a CD-ROM, or a flash drive.

In some embodiments, the provided method further includes the step of providing to the subject a diagnosis and/or the results of treatment.

### VIII. Methods for Inducing an Immune Response

In another aspect, the present disclosure provides various method for inducing an immune response in a subject. The methods generally include administering to the subject any of the retroviral vector systems, retrovirus-packaging cells, retroviruses, or virus-like particles disclosed herein, e.g., in Sections III, IV, V, and VI. For example, an immunogenic composition can be formed including any of the provided retroviral vector systems, retrovirus-packaging cells, retroviruses, or virus-like particles. The immunogenic composition can be a vaccine, and the administration of the immunogenic composition can include vaccinating the subject with the vaccine.

The disclosed immunogenic compositions can be administered using the provided method as a single dose or as multiple doses, for example, two doses administered at an interval of about one week, two weeks, three weeks, one month, about two months, about three months, about six months, or about 12 months. Other suitable dosage schedules can be determined by a medical practitioner. In some embodiments, additional compounds or medications can be co-administered to the subject. Such compounds or medications can be co-administered to, for example, alleviate signs or symptoms of the disease being treated, or to reduce side effects caused by induction of the immune response.

### IX. Pharmaceutical Compositions

In another aspect, a pharmaceutical composition is provided. The provided pharmaceutical composition includes one or more, e.g., two or more, of any of the retroviral vector systems, retrovirus-packaging cells, retroviruses, or virus-like particles disclosed herein, e.g., in Sections III, IV, V, and VI. The provided pharmaceutical compositions can, for example, better allow the retroviruses or virus-like particles disclosed herein to deliver genetic payloads in situ to a subject in need thereof.

In some embodiments, the pharmaceutical composition includes a therapeutically effective amount of a pharmaceutically acceptable excipient. In some embodiments, the pharmaceutical composition includes one or more of a diluent, adjuvant, or carrier in a formulation suitable for administration, e.g., administration to a mammal. Suitable diluents, adjuvants, or carriers can include, for example, lipids, e.g., liposomes, e.g., liposome dendrimers; liquids, such as water and oils, including those of petroleum, animal, vegetable, or synthetic origin, such as peanut oil, soybean oil, mineral oil, sesame oil and the like; gum acacia; gelatin; starch paste; talc; keratin; colloidal silica; urea; and the like. Additional examples of suitable diluents include distilled water, buffered water, physiological saline, PBS, Ringer's solution, dextrose solution, and Hank's solution. The pharmaceutical compositions can also include additional substances to approximate physiological conditions, such as pH adjusting and buffering agents, toxicity adjusting agents, wetting agents, and detergents. In addition, auxiliary, thickening, lubricating, and coloring agents can alternatively or additionally be used. Pharmaceutical compositions can be formulated into preparations in solid, semisolid, liquid, or gaseous forms, such as tablets, capsules, powders, granules, ointments, solutions, suppositories, injections, inhalants, gels, microspheres, and aerosols.

The provided pharmaceutical composition can also include any of a variety of stabilizing agents, such as an antioxidant for example. When the pharmaceutical composition includes a polypeptide, the polypeptide can be complexed with various well-known compounds that enhance the in vivo stability of the polypeptide, or otherwise enhance its pharmacological properties (e.g., increase the half-life of the polypeptide, reduce its toxicity, and/or enhance solubility or uptake). Examples of such modifications or complexing agents include sulfate, gluconate, citrate, and phosphate. The nucleic acids or polypeptides of a composition can also be complexed with molecules that enhance their in vivo attributes. Such molecules include, for example, carbohydrates, polyamines, amino acids, other peptides, ions (e.g., sodium, potassium, calcium, magnesium, manganese), and lipids.

### X. Exemplary Embodiments

The following embodiments are contemplated. All combinations of features and embodiment are contemplated.

Embodiment 1: A retroviral vector system comprising: an envelope plasmid encoding a viral membrane fusion protein of a retrovirus; a packaging plasmid encoding Gag-Pol proteins of the retrovirus; and a transfer plasmid comprising one or more genes of interest for transfer from the retrovirus to a target cell; wherein one or both of the envelope plasmid and the packaging plasmid further encodes a binding moiety that directly or indirectly binds a surface feature of the target cell.

Embodiment 2: An embodiment of embodiment 1, wherein the viral membrane fusion protein is an engineered variant of a wild-type viral membrane fusion protein.

Embodiment 3: An embodiment of embodiment 2, wherein the engineered variant does not bind to a cognate binding partner of the wild-type viral membrane fusion protein.

Embodiment 4: An embodiment of embodiment 2 or 3, wherein the engineered variant is a truncated mutant of the wild-type viral membrane fusion protein.

Embodiment 5: An embodiment of any one of embodiments 1-4, wherein the binding moiety comprises an extramembrane domain and a transmembrane domain.

Embodiment 6: An embodiment of any one of embodiments 1-5, wherein the envelope plasmid encodes the binding moiety.

Embodiment 7: An embodiment of any one of embodiments 1-5, wherein the packaging plasmid encodes the binding moiety.

Embodiment 8: An embodiment of any one of embodiments 1-7, wherein the binding moiety binds a ligand comprising an antibody, an antibody mimetic, a single-chain variable fragment (scFv), or a derivate or fragment thereof, and wherein the ligand binds the surface feature of the target cell.

Embodiment 9: An embodiment of embodiment 8, wherein the binding moiety comprises a fluorescein isothiocyanate (FITC)-binding domain, and wherein the ligand is FITC-conjugated.

Embodiment 10: An embodiment of embodiment 9, wherein the binding moiety comprises an anti-fluorescein scFv or fluorescein-binding anticalin.

Embodiment 11: An embodiment of embodiment 9 or 10, wherein the ligand is an FITC-conjugated antibody.

Embodiment 12: An embodiment of embodiment 8, wherein the binding moiety comprises a biotin-binding domain, and wherein the ligand is biotinylated.

Embodiment 13: An embodiment of embodiment 12, wherein the binding moiety comprises an anti-biotin scFv, a polyclonal anti-biotin antibody, a monoclonal anti-biotin antibody, biotin-binding anticalin, or an avidin-family protein,

Embodiment 14: An embodiment of embodiment 12 or 13, wherein the ligand is a biotinylated antibody,

Embodiment 15: An embodiment of any one of embodiments 8-14, wherein the surface feature of the target cell comprises CD3, CD4, CD7, CD8, CD19, CD20, CD56, CD71, or CTLA4.

Embodiment 16: An embodiment of any one of embodiments 1-7, wherein the binding moiety directly binds the surface feature of the target cell.

Embodiment 17: An embodiment of embodiment 16, wherein the binding moiety comprises an scFv or a nanobody.

Embodiment 18: An embodiment of embodiment 16 or 17, wherein the surface feature of the target cell comprises CD19 or CD7.

Embodiment 19: An embodiment of any one of embodiments 16-18, wherein the target cell is a T cell, a B cell, a natural killer (NK) cell, an astrocyte, a dendritic cell (DC), or a monocyte.

Embodiment 20: An embodiment of any one of embodiments 1-19, wherein the retrovirus is an alpharetrovirus, a gammaretrovirus, or a lentivirus.

Embodiment 21: An embodiment of any one of embodiments 1-20, wherein the viral membrane fusion protein is the vesicular stomatitis virus G (VSV-G) protein, is from feline endogenous virus RD114, or is from baboon endogenous virus BaEV.

Embodiment 22: An embodiment of any one of embodiments 1-21, wherein the retrovirus is an integrase-deficient retrovirus.

Embodiment 23: An embodiment of any one of embodiments 1-22, wherein the one or more genes of interest encode a chimeric antigen receptor (CAR), a switch receptor, or a derivative or fragment thereof.

Embodiment 24: An embodiment of any one of embodiments 1-23, wherein the one or more genes of interest encode a CRISPR-associated (Cas) protein, a zinc finger nuclease (Z. FN). a transcription activator-like effector nuclease (TALEN), a meganuclease, an RNA-binding protein (RBP), a recombinase, a flippase, a base editor, a prime editor, nuclease-impaired Cas, nuclease-dead Cas, an epigenome editor, a transcriptional modifier (CRISPRa/i), a transposase. an Argonaute (Ago) protein, an adenosine deaminase acting on RNA (ADAR), a Pumilio RNA-binding family (PUF) protein, a homing endonuclease, or a derivative or fragment thereof.

Embodiment 25: An embodiment of any one of embodiments 1-24, wherein the one or more genes of interest encode a fluorescent protein, an antibiotic resistance gene, or a derivative or fragment thereof.

Embodiment 26: A retrovirus-packaging cell comprising the retroviral vector system of any one of embodiments 1-25.

Embodiment 27: A retrovirus comprising: a viral membrane fusion protein; a viral genome comprising one or more genes of interest for transfer from the retrovirus to a target cell; and a binding moiety that binds a ligand comprising an antibody, an antibody mimetic, a single-chain variable fragment (scFv), or a derivate or fragment thereof, wherein the ligand binds a surface feature of the target cell.

Embodiment 28: An embodiment of embodiment 27, wherein the viral membrane fusion protein is an engineered variant of a wild-type viral membrane fusion protein.

Embodiment 29: An embodiment of embodiment 28, wherein the engineered variant does not bind to a cognate binding partner of the wild-type viral membrane fusion protein.

Embodiment 30: An embodiment of embodiment 28 or 29, wherein the engineered variant is a truncated mutant of the wild-type viral membrane fusion protein.

Embodiment 31: An embodiment of any one of embodiments 27-30, wherein the binding moiety comprises an extramembrane domain and a transmembrane domain.

Embodiment 32: An embodiment of embodiment 29 or 30, wherein the engineered variant of the wild-type viral membrane fusion protein comprises the binding moiety.

Embodiment 33: An embodiment of any one of embodiments 27-32, wherein the binding moiety comprises a fluorescein isothiocyanate (FITC)-binding domain, and wherein the ligand is FITC-conjugated.

Embodiment 34: An embodiment of embodiment 33, wherein the binding moiety comprises an anti-fluorescein scFv or fluorescein-binding anticalin.

Embodiment 35: An embodiment of embodiment 33 or 34, wherein the ligand is an FITC-conjugated antibody.

Embodiment 36: An embodiment of any one of embodiments 27-32, wherein the binding moiety comprises a biotin-binding domain, and wherein the ligand is biotinylated.

Embodiment 37: An embodiment of embodiment 36, wherein the binding moiety comprises an anti-biotin scFv, biotin-binding anticalin, or an avidin-family protein.

Embodiment 38: An embodiment of embodiment 36 or 37, wherein the ligand is a biotinylated antibody.

Embodiment 39: An embodiment of any one of embodiments 27-38, wherein the surface feature of the target cell comprises CD3, CD4, CD7, CD8, CD19, CD20, CD56, CD71, or CTLA4.

Embodiment 40: An embodiment of any one of embodiments 27-39, wherein the retrovirus is an alpharetrovirus, a gammaretrovirus, or a lentivirus.

Embodiment 41: An embodiment of any one of embodiments 27-40, wherein the viral membrane fusion protein is the vesicular stomatitis virus G (VSV-G) protein, is from feline endogenous virus RD114, or is from baboon endogenous virus BaEV.

Embodiment 42: An embodiment of any one of embodiments 27-41, wherein the retrovirus is an integrase-deficient retrovirus.

Embodiment 43: An embodiment of any one of embodiments 27-42, wherein the one or more genes of interest encode a chimeric antigen receptor (CAR), a switch receptor, or a derivative or fragment thereof.

Embodiment 44: An embodiment of any one of embodiments 27-43, wherein the one or more genes of interest encode a CRISPR-associated (Cas) protein, a zinc finger nuclease (ZFN), a transcription activator-like effector nuclease (TALEN), a meganuclease, an RNA-binding protein (RBP), a recombinase, a flippase, a base editor, a prime editor, nuclease-impaired Cas, nuclease-dead Cas, an epigenome editor, a transcriptional modifier (CRISPRa/i), a transposase. an Argonaute (Ago) protein, an adenosine deaminase acting on RNA (ADAR), a Pumilio RNA-binding family (PUF) protein, a homing endonuclease, or a derivative or fragment thereof.

Embodiment 45: A virus-like particle comprising a binding moiety that binds a ligand comprising an antibody, an antibody mimetic, a single-chain variable fragment (scFv), or a derivate or fragment thereof, wherein the ligand binds a surface feature of a target cell.

Embodiment 46: An embodiment of embodiment 45, wherein the binding moiety comprises a fluorescein isothiocyanate (FITC)-binding domain, and wherein the ligand is FITC-conjugated.

Embodiment 47: An embodiment of embodiment 46, wherein the binding moiety comprises an anti-fluorescein scFv or fluorescein-binding anticalin.

Embodiment 48: An embodiment of embodiment 46 or 47, wherein the ligand is an FITC-conjugated antibody, Embodiment 49: An embodiment of embodiment 45, wherein the binding moiety comprises a biotin-binding domain, and wherein the ligand is biotinylated.

Embodiment 50: An embodiment of embodiment 49, wherein the binding moiety comprises an anti-biotin scFv, biotin-binding anticalin, or an avidin-family protein.

Embodiment 51: An embodiment of embodiment 49 or 50, wherein the ligand is a biotinylated antibody.

Embodiment 52: An embodiment of any one of embodiments 45-51, wherein the surface feature of the target cell comprises CD3, CD4, CD7, CD8, CD19, CD20, CD56, CD71, or CTLA4.

Embodiment 53: An embodiment of any one of embodiments 45-52, comprising a CRISPR-associated (Cas) protein, a zinc finger nuclease (ZFN). a transcription activator-like effector nuclease (TALEN), a meganuclease, an RNA-binding protein (RBP), a recombinase, a flippase, a base editor, a prime editor, nuclease-impaired Cas, nuclease-dead Cas, an epigenome editor, a transcriptional modifier (CRISPRa/i), a transposase. an Argonaute (Ago) protein, an adenosine deaminase acting on RNA (ADAR), a Pumilio RNA-binding family (PUF) protein, a homing endonuclease, or a derivative or fragment thereof.

Embodiment 54: A method for producing the retroviral vector system of any one of embodiments 1-25, the method comprising transfecting a host cell with the retroviral vector system of any one of embodiments 1-25.

Embodiment 55: A method for preventing or treating a disease in a subject, the method comprising administering to the subject the retroviral vector system of any one of embodiments 1-25, the retrovirus-packaging cell of embodiment 26, the retrovirus of any one of embodiments 27-44, or the virus-like particle of any one of embodiments 45-53.

Embodiment 56: An embodiment of embodiment 55, wherein the disease comprises a genetic disorder.

Embodiment 57: An embodiment of embodiment 56, wherein the disease comprises a cancer.

Embodiment 58: An embodiment of embodiment 57, wherein the cancer comprises a solid tumor.

Embodiment 59: An embodiment of embodiment 55, wherein the disease comprises an infection

Embodiment 60: An embodiment of embodiment 59, wherein the infection is associated with a biomedical device implant of the subject.

Embodiment 61: A method for repairing or regenerating a damaged or aged tissue of a subject, the method comprising administering to the subject the retroviral vector system of any one of embodiments 1-25, the retrovirus-packaging cell of embodiment 26, the retrovirus of any one of embodiments 27-44, or the virus-like particle of any one of embodiments 45-53, wherein the target cell comprises an induced pluripotent stem cell, an embryonic stem cell, an adult stem cell, a mesenchymal stem cell, or a progenitor cell.

Embodiment 62: An embodiment of embodiment 61, wherein the damaged or aged tissue comprises muscle cells, neural cells, or pancreatic cells.

### EXAMPLES

The present disclosure will be better understood in view of the following non-limiting examples. The following examples are intended for illustrative purposes only and do not limit in any way the scope of the present invention

### Example 1. Transduction of Lymphocytes by a Retrovirus With a Cell Binding Domain

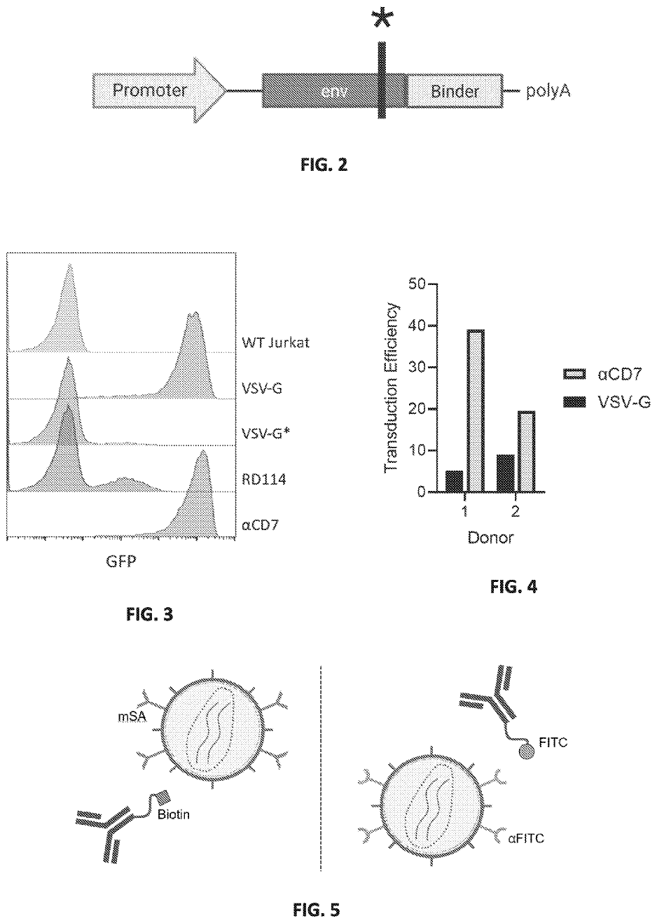

A retrovirus in accordance with a provided embodiment was created having a CD7-targeting nanobody as a cell binding domain. Wild-type Jurkat cells that do not natively express GFP (FIG. 3, top row) were transduced with a traditional lentivirus having VSV-G envelope protein and encoding an expression cassette for GFP. The cells transduced with the traditional lentivirus exhibited GFP activity (FIG. 3, second row). The VSV-G of the lentivirus was then mutated to ablate its natural binding ability. The mutated lentivirus was unable to effectively transduce the Jurkat cells (FIG. 3, third row). A feline endogenous virus pseudotyped retrovirus (RD114) was also unable to transduce the cells (FIG. 3, fourth row). The provided retrovirus with the CD7 cell binding domain, however, produced transduction results similar to that seen with the traditional lentivirus (FIG. 3, bottom row). These results demonstrate effective transduction using the retrovirus materials and methods disclosed herein.

### Example 2. Transduction of NK Cells by a Retrovirus With a Cell Binding Domain

Natural killer (NK) cells from two donors were engineering ex vivo with a chimeric receptor protein based on Toll Like Receptor 5 using two different retroviruses. The first retrovirus, a traditional lentivirus having VSV-G envelope protein, exhibited a transduction efficiency of less than 10% for each of the two donor populations of NK cells (FIG. 4). The second retrovirus, a retrovirus having a CD7-binding cell binding domain in accordance with a provided embodiment, exhibited 2-fold to 8-fold higher transduction efficiency than seen with the traditional lentivirus (FIG. 4). These results demonstrate the ability of the retrovirus materials and methods disclosed herein to effectively transduce therapeutically important cell types not easily transduced with previous retrovirus approaches.

### Example 3. Transduction by a Retrovirus With an Antibody Binding Domain

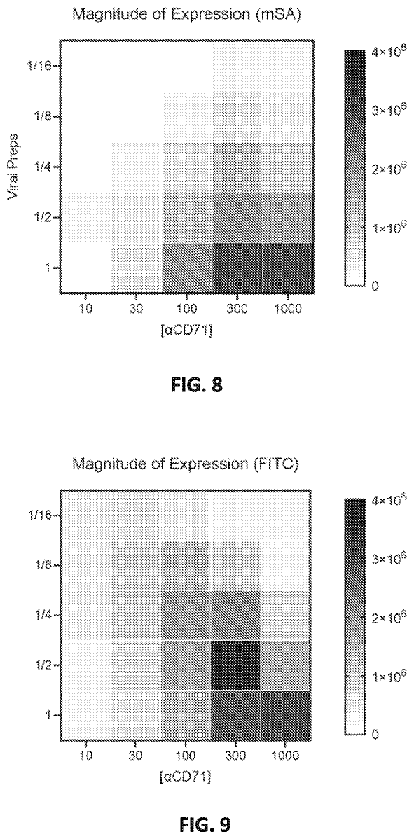

Universal pseudotyped retroviruses in accordance with provided embodiments were generated by swapping the binding moiety with a monomeric streptavidin (mSA) biotin-binding domain, or with a FITC-binding antibody domain (αFITC). The retroviruses each caried an mCherry expression cassette. Jurkat cells were then exposed to different concentrations of the retroviruses, where the retroviruses were bound to different concentrations of a CD71 antibody (αCD71). Results demonstrated that both the FITC-binding retrovirus (FIG. 6) and the biotin-binding retrovirus (FIG. 7) transduced the Jurkat cells when bound to the antibody. Further, both provided retroviruses can transduce Jurkat cells multiple times, a feature indicative of lentivirus. Additional experiments demonstrated that optimizing both the amount of virus and concentration of an antibody can increase the expression of the delivered genetic payload, presumably by multiple integration events (FIGS. 8 and 9).

### Example 4. Targeting of Different Cell Markers and Epitopes With a Universal Pseudotyped Retrovirus

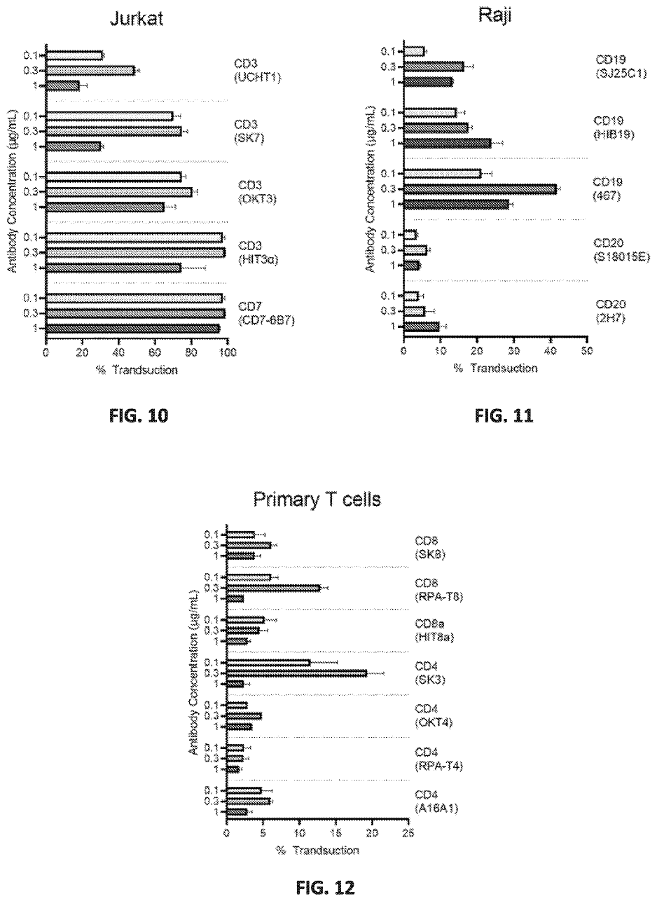

A universal pseudotyped retrovirus was created in accordance with a provided embodiment, where the retrovirus included a FITC-binding scFv and an mCherry expression cassette. The retrovirus was used to transduce Jurkat cells (FIG. 10) engineered with antibodies against lymphocyte markers (CD7) and T cell markers (CD3), Raji cells (FIG. 11) with B cell markers (CD19 and CD20), and primary human T cells (FIG. 12) with subtype markers (CD4 and CD8). Each antibody was tested at 3 concentrations and many antibody clones were tested for each marker (FIGS. 10-12). The results demonstrated that universal pseudotyped retrovirus can be effective against multiple targets and multiple epitopes on those targets.

### Example 5. Selective Cell Targeting With a Universal Pseudotyped Retrovirus

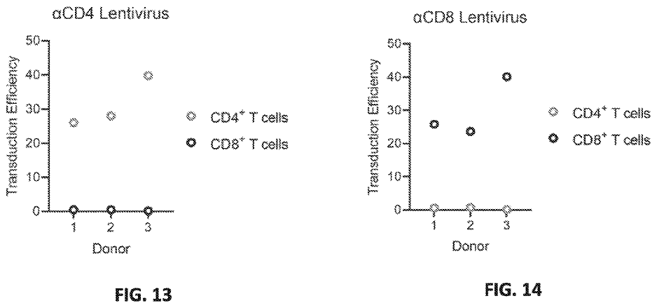

A universal pseudotyped retrovirus was produced in accordance with a provided embodiment, where the retrovirus included a FITC-binding scFv and an mCherry expression cassette. Portions of the retrovirus were conjugated to either a CD4 (FIG. 13) or CD8 (FIG. 14) antibody, and delivered to primary human T cells containing both CD4-only and CD8-only T cells. In each of three tested donor populations, the retrovirus effectively transduced only their intended target, with virtually no observed off-target cell engineering (FIGS. 13 and 14). These results demonstrate that universal pseudotyped retrovirus as disclosed herein can selectively engineer specific cell populations in a mixture.

### Example 6. Delivery of Protein Demonstrating in Vivo Activity to Mouse Models

A targeted pseudotyped lentivirus in accordance with a provided embodiment is generated using standard methods. Briefly, a transfer plasmid containing the genetic payload of interest, a packaging plasmid, and modified envelope plasmid, as described in Section III, are transfected into HEK293T cells. The modified envelope plasmid is mutated to ablate the binding properties of VSV-G and engineered to encode a binding moiety. The binding moiety can be one configured or selected to bind a cell marker directly, or can be one configured or selected to bind a moiety contained on a secondary binder such as a biotin-or FITC-conjugated antibody. The virus is isolated and purified. If a secondary binder, i.e., a ligand, is used, then the purified virus and secondary binder are incubated together for a suitable period of time prior to administration.

In particular examples, the payload of interest is GFP, and the binding moiety is either a CD7 nanobody (nbCD7) or a monomeric streptavidin (mSA). In the case of mSA, the virus is incubated with biotin conjugated CD7 antibody for 30 min. In both cases, the virus is injected into mice intravenously. About one week later, the spleens and peripheral blood of the mice are taken and processed to isolate immune cells (splenocytes or peripheral blood mononuclear cells respectively). Cells are stained for CD7, and the degree of GFP in the CD7+ fraction is quantified to confirm on-target efficiency, while the GFP in the CD7-fraction is quantified to show off-target effects.

Although the foregoing disclosure has been described in some detail by way of illustration and example for purpose of clarity of understanding, one of skill in the art will appreciate that certain changes and modifications within the spirit and scope of the disclosure may be practiced, e.g., within the scope of the appended claims. It should also be understood that aspects of the disclosure and portions of various recited embodiments and features can be combined or interchanged either in whole or in part. In the foregoing descriptions of the various embodiments, those embodiments which refer to another embodiment may be appropriately combined with other embodiments as will be appreciated by one of skill in the art. Furthermore, those of ordinary skill in the art will appreciate that the foregoing description is by way of example only, and is not intended to limit the disclosure. In addition, each reference provided herein is incorporated by reference in its entirety for all purposes to the same extent as if each reference was individually incorporated by reference.

What is claimed is:

1. A retroviral vector system comprising:
an envelope plasmid encoding a viral membrane fusion protein of a retrovirus;
a packaging plasmid encoding Gag-Pol proteins of the retrovirus; and
a transfer plasmid comprising one or more genes of interest for transfer from the retrovirus to a target cell;
wherein one or both of the envelope plasmid and the packaging plasmid further encodes a binding moiety that directly or indirectly binds a surface feature of the target cell.

2. The retroviral vector system of claim 1, wherein the viral membrane fusion protein is an engineered variant of a wild-type viral membrane fusion protein.

3. The retroviral vector system of claim 2, wherein the engineered variant does not bind to a cognate binding partner of the wild-type viral membrane fusion protein.

4. The retroviral vector system of claim 2 or 3, wherein the engineered variant is a truncated mutant of the wild-type viral membrane fusion protein.

5. The retroviral vector system of any one of claims 1-4, wherein the binding moiety comprises an extramembrane domain and a transmembrane domain.

6. The retroviral vector system of any one of claims 1-5, wherein the envelope plasmid encodes the binding moiety.

7. The retroviral vector system of any one of claims 1-5, wherein the packaging plasmid encodes the binding moiety.

8. The retroviral vector system of any one of claim 1-7, wherein the binding moiety binds a ligand comprising an antibody, an antibody mimetic, a single-chain variable fragment (scFv), or a derivate or fragment thereof, and wherein the ligand binds the surface feature of the target cell.

9. The retroviral vector system of claim 8, wherein the binding moiety comprises a fluorescein isothiocyanate (FITC)-binding domain, and wherein the ligand is FITC-conjugated.

10. The retroviral vector system of claim 9, wherein the binding moiety comprises an anti-fluorescein scFv or fluorescein-binding anticalin.

11. The retroviral vector system of claim 9 or 10, wherein the ligand is an FITC-conjugated antibody.

12. The retroviral vector system of claim 8, wherein the binding moiety comprises a biotin-binding domain, and wherein the ligand is biotinylated.

13. The retroviral vector system of claim 12, wherein the binding moiety comprises an anti-biotin scFv, a polyclonal anti-biotin antibody, a monoclonal anti-biotin antibody, biotin-binding anticalin, or an avidin-family protein.

14. The retroviral vector system of claim 12 or 13, wherein the ligand is a biotinylated antibody.

15. The retroviral vector system of any one of claims 8-14, wherein the surface feature of the target cell comprises CD3, CD4, CD7, CD8, CD19, CD20, CD56, CD71, or CTLA4.

16. The retroviral vector system of any one of claims 1-7, wherein the binding moiety directly binds the surface feature of the target cell.

17. The retroviral vector system of claim 16, wherein the binding moiety comprises an scFv or a nanobody.

18. The retroviral vector system of claim 16 or 17, wherein the surface feature of the target cell comprises CD19 or CD7.

19. The retroviral vector system of any one of claims 1-18, wherein the target cell is a T cell, a B cell, a natural killer (NK) cell, an astrocyte, a dendritic cell (DC), or a monocyte.

20. The retroviral vector system of any one of claims 1-19, wherein the retrovirus is an alpharetrovirus, a gammaretrovirus, or a lentivirus.

21. The retroviral vector system of any one of claims 1-20, wherein the viral membrane fusion protein is the vesicular stomatitis virus G (VSV-G) protein, is from feline endogenous virus RD114, or is from baboon endogenous virus BaEV.

22. The retroviral vector system of any one of claims 1-21, wherein the retrovirus is an integrase-deficient retrovirus.

23. The retroviral vector system of any one of claims 1-22, wherein the one or more genes of interest encode a chimeric antigen receptor (CAR), a switch receptor, or a derivative or fragment thereof.

24. The retroviral vector system of any one of claims 1-23, wherein the one or more genes of interest encode a CRISPR-associated (Cas) protein, a zinc finger nuclease (ZFN), a transcription activator-like effector nuclease (TALEN), a meganuclease, an RNA-binding protein (RBP), a recombinase, a flippase, a base editor, a prime editor, nuclease-impaired Cas, nuclease-dead Cas, an epigenome editor, a transcriptional modifier (CRISPRa/i), a transposase. an Argonaute (Ago) protein, an adenosine deaminase acting on RNA (ADAR), a Pumilio RNA-binding family (PUF) protein, a homing endonuclease, or a derivative or fragment thereof.

25. The retroviral vector system of any one of claims 1-24, wherein the one or more genes of interest encode a fluorescent protein, an antibiotic resistance gene, or a derivative or fragment thereof.

26. A retrovirus-packaging cell comprising the retroviral vector system of any one of claims 1-25.

27. A retrovirus comprising:
a viral membrane fusion protein;
a viral genome comprising one or more genes of interest for transfer from the retrovirus to a target cell; and
a binding moiety that binds a ligand comprising an antibody, an antibody mimetic, a single-chain variable fragment (scFv), or a derivate or fragment thereof, wherein the ligand binds a surface feature of the target cell.

28. The retrovirus of claim 27, wherein the viral membrane fusion protein is an engineered variant of a wild-type viral membrane fusion protein.

29. The retrovirus of claim 28, wherein the engineered variant does not bind to a cognate binding partner of the wild-type viral membrane fusion protein.

30. The retrovirus of claim 28 or 29, wherein the engineered variant is a truncated mutant of the wild-type viral membrane fusion protein.

31. The retrovirus of any one of claims 27-30, wherein the binding moiety comprises an extramembrane domain and a transmembrane domain.

32. The retrovirus of any one of claims 29 or 30, wherein the engineered variant of the wild-type viral membrane fusion protein comprises the binding moiety.

33. The retrovirus of any one of claims 27-32, wherein the binding moiety comprises a fluorescein isothiocyanate (FITC)-binding domain, and wherein the ligand is FITC-conjugated.

34. The retrovirus of claim 33, wherein the binding moiety comprises an anti-fluorescein scFv or fluorescein-binding anticalin.

35. The retrovirus of claim 33 or 34, wherein the ligand is an FITC-2 conjugated antibody.

36. The retrovirus of any one of claims 27-32, wherein the binding moiety comprises a biotin-binding domain, and wherein the ligand is biotinylated.

37. The retrovirus of claim 36, wherein the binding moiety comprises an anti-biotin scFv, biotin-binding anticalin, or an avidin-family protein.

38. The retrovirus of claim 36 or 37, wherein the ligand is a biotinylated antibody.

39. The retrovirus of any one of claims 27-38, wherein the surface feature of the target cell comprises CD3, CD4, CD7, CD8, CD19, CD20, CD56, CD71, or CTLA4.

40. The retrovirus of any one of claims 27-39, wherein the retrovirus is an alpharetrovirus, a gammaretrovirus, or a lentivirus.

41. The retrovirus of any one of claims 27-40, wherein the viral membrane fusion protein is the vesicular stomatitis virus G (VSV-G) protein, is from feline endogenous virus RD114, or is from baboon endogenous virus BaEV.

42. The retrovirus of any one of claims 27-41, wherein the retrovirus is an integrase-deficient retrovirus.

43. The retrovirus of any one of claims 27-42, wherein the one or more genes of interest encode a chimeric antigen receptor (CAR), a switch receptor, or a derivative or fragment thereof.

44. The retrovirus of any one of claims 27-43, wherein the one or more genes of interest encode a CRISPR-associated (Cas) protein, a zinc finger nuclease (ZFN), a transcription activator-like effector nuclease (TALEN), a meganuclease, an RNA-binding protein (RBP), a recombinase, a flippase, a base editor, a prime editor, nuclease-impaired Cas, nuclease-dead Cas, an epigenome editor, a transcriptional modifier (CRISPRa/i), a transposase. an Argonaute (Ago) protein, an adenosine deaminase acting on RNA (ADAR), a Pumilio RNA-binding family (PUF) protein, a homing endonuclease, or a derivative or fragment thereof.

45. A virus-like particle comprising a binding moiety that binds a ligand comprising an antibody, an antibody mimetic, a single-chain variable fragment (scFv), or a derivate or fragment thereof, wherein the ligand binds a surface feature of a target cell.

46. The virus-like particle of claim 45, wherein the binding moiety comprises a fluorescein isothiocyanate (FITC)-binding domain, and wherein the ligand is FITC-conjugated.

47. The virus-like particle of claim 46, wherein the binding moiety comprises an anti-fluorescein scFv or fluorescein-binding anticalin.

48. The virus-like particle of claim 46 or 47, wherein the ligand is an FITC-conjugated antibody.

49. The virus-like particle of claim 45, wherein the binding moiety comprises a biotin-binding domain, and wherein the ligand is biotinylated.

50. The virus-like particle of claim 49, wherein the binding moiety comprises an anti-biotin scFv, biotin-binding anticalin, or an avidin-family protein.

51. The virus-like particle of claim 49 or 50, wherein the ligand is a biotinylated antibody.

52. The virus-like particle of any one of claims 45-51, wherein the surface feature of the target cell comprises CD3, CD4, CD7, CD8, CD19, CD20, CD56, CD71, or CTLA4.

53. The virus-like particle of any one of claims 45-52, comprising a CRISPR-associated (Cas) protein, a zinc finger nuclease (ZFN). a transcription activator-like effector nuclease (TALEN), a meganuclease, an RNA-binding protein (RBP), a recombinase, a flippase, a base editor, a prime editor, nuclease-impaired Cas, nuclease-dead Cas, an epigenome editor, a transcriptional modifier (CRISPRa/i), a transposase. an Argonaute (Ago) protein, an adenosine deaminase acting on RNA (ADAR), a Pumilio RNA-binding family (PUF) protein, a homing endonuclease, or a derivative or fragment thereof.

54. A method for producing the retroviral vector system of any one of claims 1-25, the method comprising transfecting a host cell with the retroviral vector system of any one of claims 1-25.

55. A method for preventing or treating a disease in a subject, the method comprising administering to the subject the retroviral vector system of any one of claims 1-25, the retrovirus-packaging cell of claim 26, the retrovirus of any one of claims 27-44, or the virus-like particle of any one of claims 45-53.

56. The method of claim 55, wherein the disease comprises a genetic disorder.

57. The method of claim 55, wherein the disease comprises a cancer.

58. The method of claim 57, wherein the cancer comprises a solid tumor.

59. The method of claim 55, wherein the disease comprises an infection.

60. The method of claim 59, wherein the infection is associated with a biomedical device implant of the subject.

61. A method for repairing or regenerating a damaged or aged tissue of a subject, the method comprising administering to the subject the retroviral vector system of any one of claims 1-25, the retrovirus-packaging cell of claim 26, the retrovirus of any one of claims 27-44, or the virus-like particle of any one of claims 45-53, wherein the target cell comprises an induced pluripotent stem cell, an embryonic stem cell, an adult stem cell, a mesenchymal stem cell, or a progenitor cell.

62. The method of claim 61, wherein the damaged or aged tissue comprises muscle cells, neural cells, or pancreatic cells.
